(19) United States
(12) Patent Application Publication
(10) Pub. No.: US 2007/0294107 A1
(43) Pub. Date: Dec. 20, 2007

(54) Uses Of Endopeptidases In Treatment Of Disease

(76) Inventors: Geert W. Schmid-Schonbein, Del Mar, CA (US); Frank A. Delano, San Diego, CA (US)

Correspondence Address: FISH & ASSOCIATES, PC;ROBERT D. FISH, 2603 Main Street, Suite 1050, Irvine, CA, 92614-6232, US

(21) Appl. No.: 11/850,169

(22) Filed: Sep. 5, 2007

Related U.S. Application Data
(63) Continuation-in-part of application No. 11/638,432, filed on Dec. 14, 2006.
(60) Provisional application No. 60/750,356, filed on Dec. 15, 2005.

(51) Int. Cl. G06Q 50/00; A61K 31/65; A61P 29/00; A61P 3/10; A61P 9/12

(52) U.S. Cl. 705/2; 514/152

(57) ABSTRACT

Compounds and methods of treatment of inflammation are presented that serve to enhance MMP inhibition to block elevation of MMP activity in various disease conditions. Such treatment leads to normalization of function, blockade of the elevated levels of oxygen free radical production in the microcirculation, normalization of the elevated plasma levels of MMP 2, 9 activity, normalization of immune cell counts, and reduced apoptosis. Also included are methods of deriving value from increased sales of public domain compositions following recognition of their usefulness in treating the diseases. Of particular interest is use of doxycycline to treat diabetes and hypertension.

This application is a Continuation-in-Part (CIP) of U.S. patent application Ser. No. 11/638,432, filed Dec. 14, 2006, which claims priority to U.S. Provisional Patent Application Ser. No. 60/750,356, filed Dec. 15, 2005, the contents of which are hereby incorporated by reference herein in their entirety.

### GOVERNMENT INTERESTS

This invention was made with U.S. Government support under National Institute of Health (NIH) Grant No. HL10881. The government has certain rights in this invention.

### FIELD OF THE INVENTION

The present invention relates to treatment of diseases having an etiology related to matrix metalloprotein (MMP) induced inflammation, and to commercially desirable methods for same.

### BACKGROUND OF THE INVENTION

An increasing number of cardiovascular complications have been shown to be accompanied by markers of inflammation. These conditions include not only acute complications, such as cardiac or cerebral ischemia, atherosclerosis, pain, or the metabolic syndrome, but also chronic degenerative conditions such as ocular disease, venous disease, arterial hypertension, diabetes, arthritis, as well as the process of aging. The inflammatory cascade involves a sequence of stereotypic events that can be observed in all organs and in many situations is observed in the microcirculation years before clinical manifestations. Similar statements can be made for other, seemingly unrelated diseases, such as diabetes.

It has become evident in recent work that interventions against individual steps in the inflammatory cascade (e.g., blockade of oxygen free radicals, cytokines, membrane adhesion molecules, blockade of the complement and thrombotic cascades or production of lipid mediators) leads to a mixed level of success, in some cases despite preclinical evidence suggesting that the inflammatory process can be attenuated.

About a decade ago, a different approach was sought to analyze the inflammatory cascade. Instead of looking exclusively at individual steps and/or markers for the inflammatory cascade, the new idea was to identify trigger mechanisms for inflammation. This new approach has lead to a fundamental new understanding of the origin of inflammation in acute situations.

An important consideration to control the level of inflammation is to identify molecular mechanisms that can trigger an inflammatory reaction. To achieve this, it is most useful to detect inflammation at an early stage. One of the ways is to study the level of cell activation in the microcirculation. Besides the study of endothelial cells and mast cells in the tissue, a number of techniques have been developed to study cell activation by use of circulating leukocytes or platelets. Markers of leukocyte activation include pseudopod formation (by actin polymerization), free radical production (detectable by use of indicators), expression of membrane adhesion molecules or detection of the level of degranulation.

These studies showed that there are a large number of mechanisms to activate cells and stimulate an inflammatory cascade. It is convenient to classify mechanisms for cell activation into several general categories:
a) Positive feedback mechanisms: There exists a class of inflammatory reactions that are mediated by direct action of plasma inflammatory stimulators (oxygen free radicals, platelet activating factor (PAF), cytokines (e.g., TNF-α, IL-I, IL-8), complement fragments, endotoxins, coagulation and fibrinolytic factors, leukotrienes, thrombin, and oxidized LDL). The list of inflammatory mediators is long, and may in part be triggered by trauma or by bacterial, viral, or fungal sources.
b) Negative feedback mechanisms: An alternative pathway for cell upregulation in the microcirculation is by depletion of anti-inflammatory factors. This list is shorter and includes nitric oxide, adenosine, glucocorticoids, and selected cytokines (e.g., IL-10).
c) Contact activation: A specialized form of cell activation by membrane contact has been proposed in the form of juxtacrine activation. A non-activated endothelial cell may be stimulated during membrane contact by an activated leukocyte and vice versa, e.g., by oxygen free radical production in the membrane contact region between the cells and by formation of platelet activating factor (PAF) and other bioactive lipids.
d) Activation by mechanotransduction: Alternative forms of cell activation due to either a shift to un-physiologically low or high fluid shear stresses acting on the endothelium or a shift in the oxygen supply to the tissue. Fluid shear serves as a control mechanism for various forms of cell activation and the expression of anti-inflammatory and pro-inflammatory genes. Inflammatory stimulators of the sort listed above can influence the fluid shear response via a cGMP mediated mechanism.
e) Activation by physical transients: Transients of gas (like oxygen, carbon dioxide, etc.) concentrations or temperature transients have the ability to stimulate cell activation irrespective of the direction of the transient (up or down) but dependent on the magnitude of the transient.

Over the lifetime of an individual, it is likely that several, if not all, of these mechanisms will at one time or another stimulate inflammation. The challenge is to identify prevailing mechanisms, and then to correlate these mechanisms with specific diseases or classes of diseases.

For example, conventional studies have shown that drug treatments serve to reduce blood pressure in hypertensives (Beta-adrenergic receptor blockers, calcium channel blockers, ACE inhibitors). Also, oxygen free radical scavenging has been shown to reduce hypertensive symptoms in the spontaneously hypertensive rat (SHR). What is needed with respect to hypertension are new approaches for identifying and controlling any related inflammatory aspects. Initial observations indicate that the heightened plasma protease activity that is observed in hypertensives (SHR) may originate from the intestines. Alternatively, the protease activity that is observed in the plasma may also be a result of synthesis from the endothelial cells (or others). Although the exact origin of the protease activity in the plasma has not been definitively determined at this point, the more relevant issue is its heightened level of activity, the effects that such heightened levels have on hypertension, and methods to treat such hypertension by directly controlling the heightened levels of protease. But even with all of this information, it is not at all obvious how to treat or prevent hypertension from an inflammatory perspective.

Thus, there is a need in the art for methods and compositions for treating diseases that are not generally regarded as being inflammatory diseases, but which have at least some inflammatory component to their etiology.

### SUMMARY OF THE INVENTION

The present invention provides methods and compositions for treating disease, and related methods of marketing and profiting from same. The diseases of greatest interest are those that are not generally regarded as being inflammatory diseases, but which have at least some inflammatory component to their etiology. Such diseases include, for example, insulin resistance and other aspects of diabetes, arterial hypertension, leukophilia, and “syndrome x”. Most typically, but not necessarily, such diseases are non-gingival diseases. Further contemplated diseases include those in which free radical production is implicated. The contemplated methods and compositions are intended to either block or reduce the inflammation, and/or de-couple the inflammation from progression of the disease.

In a first aspect of the invention, methods of treating a disease comprise (a) receiving information that the disease is associated with MMP activity, wherein the disease comprises at least one of insulin resistance, hypertension, thrombosis, and free radical production; (b) receiving information that a compound has activity as an MMP inhibitor; and (c) administering the compound in an amount estimated to be sufficient to treat the disease. The MMP inhibitor preferably comprises doxycycline, which can be advantageously formulated for oral delivery.

In a second aspect of the invention, a pharmaceutical grade compound is packaged with a first reference to activity of the compound in inhibiting an endopeptidase, and a second reference to effectiveness of the compound in treating diabetes. In preferred embodiments the endopeptidase comprises a matrix metalloproteinase, and the compound comprises at least one of doxycycline and a doxycycline derivative. In especially preferred embodiments, a compound can be advantageously labeled with: (a) a reference to at least one of anti-diabetic activity, thrombosis, hypertension activity of the compound; (b) a reference to metalloproteinase; and (c) wherein the compound is selected from the group consisting of doxycycline, Tinospora cordifolia, vitamin D, a proanthocyanidin, a catechol, a gallocatechol, and PDCT (pyrrolidine dithiocarbamate).

A third class of aspects relate to commercially desirable and heretofore unappreciated methods relating to use of a substance that may already be in the public domain, and for which either the intended activity is novel or usefulness of the substance in treating a particular disease is novel. For example, methods of increasing a value in a company are contemplated, comprising: (a) preparing a first projection of sales for a known pharmaceutically active agent; (b) receiving information that the agent may be effective in treating a degenerative disease against which the agent was not previously known to be effective; (c) preparing a second projection showing increased projected sales of the agent following receipt of the information; and (d) providing information regarding the second projection to investors of the company. The value could, for example, comprise a market capitalization, and at least one of the projections could be a next period projection.

In preferred embodiments the agent is regarded by a federal regulatory agency as having antibiotic activity or having activity as an ACE (angiotensin converting enzyme) inhibitor. In various classes of especially preferred embodiments, the agent comprises doxycycline and the disease comprises diabetes. Contemplated methods also include supporting research that investigates whether the agent is effective in treating the disease, and the effects of differing dosages. Other contemplated methods include receiving information that the agent has effectiveness in treatment of the disease when used in a dosage that is lower than a labeled dosage for the agent. Still other contemplated methods include: (a) performing a structure/function analysis of the agent; and (b) using the analysis to identify a derivative of the agent that may be effective in treating the disease.

With respect to revenue and profits, it is contemplated that one could try to pay less than a reasonable royalty (e.g. 10%) of net revenue to an entity having ownership of a patent that claims use of the agent in treating the disease. One could also sell the agent through a related company that is at least partially owned by the company. In other embodiments one could raise a sales price of the agent following the step of receiving information that the agent may be effective in treating the disease and/or increase a size of a sales force that sells the agent, following the step of receiving information that the agent may be effective in treating the disease. In still other embodiments, the step of providing information regarding the second projection could comprise including the information in an annual report and/or a quarterly report.

In a fourth aspect of the invention, methods of increasing a value in a company comprise: (a) preparing a first projection of sales for doxycycline; and (b) achieving an increased sales volume for doxycycline as a function of use of doxycyline to treat diabetes. In preferred embodiments the methods further comprise one or more of preparing a second projection showing increased projected sales of the agent, attributing the increased sales volume to the use of doxycyline to treat diabetes; and receiving information that doxycyline can be used to treat diabetes.

Therefore, methods of marketing are also contemplated that comprise (a) providing a preparation of doxycycline for use by a patient population, and (b) placing the preparation in a stream of commerce with an expectation that at least some of the preparation will be used to treat a (preferably non-gingival) disease associated with an matrix metalloproteinase (MMP) activity. Where desired, information may be provided to a medical provider and/or a patient regarding the correlation between doxycycline and the MMP activity. The expectation may arise at least in part from receiving information from a scientist involved in establishing a correlation between doxycycline and the MMP activity, from an agent reading a journal article that discusses a correlation between doxycycline and the MMP activity, and/or via subscription to a peer-reviewed journal that contains an article that discusses a correlation between doxycycline and the MMP activity. Still further, it is contemplated that placing the preparation in a stream of commerce may be performed with an understanding that treatment of the disease may include prophylactic treatment.

In a fifth aspect of the invention, methods of contributing to commercialization of a compound other than doxycycline comprise: (a) receiving information that the compound is associated with inhibition of a metalloproteinase; (b) then receiving notice that a government agency having a responsibility for protecting public health has been asked to consider approval of the doxycycline for an anti-diabetic activity as a function of the doxycycline being an metalloproteinase inhibitor; and (c) then continuing to sell the compound. Preferred embodiments include one or more of providing information to the government agency with respect to manufacture of the compound, seeking approval from the government agency to manufacture the compound, and receiving notice that the government agency has approved use of doxycycline for the anti-diabetic activity. It is especially contemplated that the compound can be selected from the group consisting of Tinospora cordifolia and vitamin D, and that the compound can be a proanthocyanidin, catechol, gallocatechol, or other is a polyphenol antioxidant. Specifically contemplated polyphenol antioxidants include green tea extract, EGCG (epigallocatechin gallate), and grape seed extract. Alternatively, it is contemplated that the compound can be PDCT (pyrrolidine dithiocarbamate). Where the compound is doxycycline, the preparation may be marketed in a package that includes a reference to a correlation between doxycycline and the MMP activity, for example, by use of a package insert. In at least some cases, such reference may be mandated by the Food and Drug Administration.

Part of the basis of the present invention stems from several past observations, and a recent discovery that have collectively now shed new light on a mechanism for hypertension and insulin resistance. The evidence indicates that hypertensives suffer from multiple defects, including a defect in vascular leukocyte adhesion, shear stress response and insulin transport, in addition to a defect in arteriolar tone with superoxide overproduction and inadequate nitric oxide formation. It has been shown that the plasma of hypertensives exhibits enhanced protease activity. This evidence suggests that membrane receptors that mediate these conditions may be cleaved by the proteases and protease inhibition will prevent this effect.

### BRIEF DESCRIPTION OF THE DRAWINGS

FIG. 1 shows MMP2, 9 activity detected by fluorescent intensity (arrows) derived by cleavage of the fluorescent substrate indicator.

FIG. 2 shows bright field micrograph of the rat mesentery microcirculation with NBT formazan deposits.

FIG. 3 shows a micrograph of fresh circulating leukocytes after labeling with CD18 integrin antibody against the extracellular domain.

Various objects, features, aspects and advantages of the present invention will become more apparent from the following detailed description of preferred embodiments of the invention, along with the accompanying drawings in which like numerals represent like components.

### DETAILED DESCRIPTION

Support for various aspects of the claimed inventions derives from a series of studies that uncovered a unique and previously unrecognized mechanism of inflammatory mediator production involving the powerful pancreatic enzymes in the intestine. Much of the basic physiology is already known. These enzymes, fully activated and concentrated in the lumen of the intestine, are a key component of normal digestion. They are highly concentrated and optimized to digest almost all biological tissues, including proteins, lipids, nucleotides and carbohydrates, of both mammalian and amphibian source. Digestion of the gut itself is largely prevented by compartmentalization of the activated pancreatic enzymes within the lumen of the intestine by the mucosal epithelial (brush border) cells. The limited permeability of the mucosal layer under ideal physiological conditions prevents the high molecular weight digestive enzymes from escaping into the wall of the intestine.

But under a variety of conditions, including diabetes, hypertension, and aging, this mucosal barrier may not always be as tight as desired, and digestive enzymes may escape into the interstitial space of the wall of the intestine and begin to digest the intestinal wall. In the course of this process, a largely undescribed class of powerful inflammatory mediators (derived entirely from digestion of extracellular and cellular components by pancreatic enzymes) is released. We refer to these as the “inflammatory fragments” generated by digestive enzymes. These inflammatory fragments escape into the portal venous circulation, pass into the central circulation and have the ability to reach all organs to generate an inflammatory cascade. They also escape into the intestinal lymphatics as well as directly into the peritoneum and thereby bypass the normal hepatic detoxification. We refer to this sequence of events as the “self-digestion process”.

Without wishing to be held to any particular theory or mechanism of action, we currently contemplate that at least some of the inflammation in hypertension, diabetes, and aging is due to self-digestion, with cleavage of receptors and other vascular proteins. Pancreatic digestive enzymes, especially serine proteases and lipases, are a major source for production of humoral inflammatory mediators that escape in small amounts and trigger inflammation in the microcirculation. The normal diet of purpose-bred rats, for example, contains inflammatory mediators obtained after digestion with pancreatic enzymes, so that the choice of the diet may be an important determinant for the degree of leakage of pancreatic digestive enzymes across the epithelium of the mucosal barrier. There may also exist amplification mechanisms for self-digestion facilitated by escape of digestive proteases into the circulation and into the extra-cellular matrix, which cause activation of zymogen forms of matrix metalloproteases (MMP) in remote organs and further enhancement of self-digestion, and cause insulin resistance, peripheral cardiovascular complications, and progressive tissue degeneration.

Among other things, we examined development of enzymatic activity and inflammatory markers in the presence of chronic treatment with oral serine protease and matrix Metalloprotease (MMP) inhibitors. In particular we considered the proteasellipase transport across the mucosal barrier and its activity in the wall of the intestine, in plasma, and in selected vital organs by zymography, and correlated these values with glucose kinetics, inflammatory markers (in-vivo leukocyte activation, oxygen free radical production, c-reactive proteins, cytokines), and aging indices.

This led us to contemplate treatment using a broad acting MMP inhibitor, which blocks the elevation of enzymatic activity in experimental forms of arterial hypertension. In fact, we found that such treatment leads to normalization of the SHR arterial blood pressure, blockade of the elevated levels of oxygen free radical production in the microcirculation, normalization of the free elevated levels of MMP 2, 9 (and other proteolytic and lypolytic enzyme) activity, normalization of immune cell counts, reduced apoptosis and normalization of the insulin resistance to levels of asymptomatic wild type rat strains. The treatment also serves to reduce similar parameters slightly elevated in the Wistar Kyoto Rat (WKY) rat strain.

We also found that the inhibition of MMP mitigates the symptoms of diseases rooted in inflammation: Syndrome X, hypertension, diabetes, etc. Thus, by using MMP inhibitors, we propose novel methods for treatment of such diseases. In the examples used throughout this disclosure, doxycycline is used as an exemplary MMP inhibitor for sake of simplicity. However, most of the claims are not limited to doxycycline or other synthetic or natural MMP inhibitors. Any MMP inhibitor, or other similar product, that serves to control, decrease or prevent plasma protease activity in diabetics and hypertensives, are also contemplated to assist in controlling of inflammation in the diseases discussed herein.

It should be appreciated that application of doxycycline or other MMP inhibitors to patients or populations can be in any suitable dosage form, administered using any suitable route, and administered according to any suitable protocol. Among other things this includes conventional pharmaceuticals (e.g., orally administered pill, capsule, tablet, caplet etc.) and alternative forms such as nutraceuticals and addition to the diet or drinking fluids. Natural MMP inhibitors include, but are not limited to, grape seed extract, cranberry fruit extract, pomegranate fruit extract, and green tea extract, among others. Such MMP inhibitors may be added to all drinking (tap) water, in a population would entail adding a natural MMP inhibitor to certain bottled waters or juices, or in specialty drinks or other foods. The purpose would be to deliver MMP inhibitors, such as doxycycline or others, to a population in a proper dosage such that the benefits of its anti-inflammatory properties are gained without causing an excess in MMP inhibitor intake.

It should be particularly noted that doxycycline has found several therapeutic uses that received FDA approval. Most prominently, use of doxycycline was approved as therapeutic agent in the treatment of certain bacterial infections, as prophylactic agent for malaria, and also as an adjunct to dental scaling and root planning. However, regulatory approval was not granted for doxycycline uses associated with MMP inhibition, and to the best of the inventors' knowledge, no applications for such approval were filed with the FDA, likely due to undesired anti-biotic side effects and possibly also concerns associated with toxicity and long-term exposure. Such considerations have led to the development and successful testing of numerous non-doxycycline tetracycline compounds, for which various trials are now anticipated (e.g., osteoporosis, arthritis, respiratory and cardiovascular diseases).

Based on the above and other considerations (e.g., low-dose use in the range of between about 1-5 mg/dosage unit, 5-20 mg/dosage unit, 20-40 mg/dosage unit, and in less preferred cases 40-80 mg/dosage unit, or even higher), the inventors now contemplate that despite the apparent abandonment of use (from an economic, clinical, scientific point of view) of doxycycline as an MMP inhibitor in therapeutic compositions, doxycycline containing preparations can be placed in the stream of commerce with an expectation that at least some of the preparation will be used to treat a (typically non-gingival) disease associated with an matrix metalloproteinase (MMP) activity. Such expectation may be based on information from one or more scientists that are involved in establishing a correlation between doxycycline and the MMP activity, from review of various publications (e.g., peer reviewed journal) by an agent or other officer of a supplier, marketer, and/or producer of doxycycline containing preparations, wherein the supplier, marketer, and/or producer may or may not subscribe to the publication.

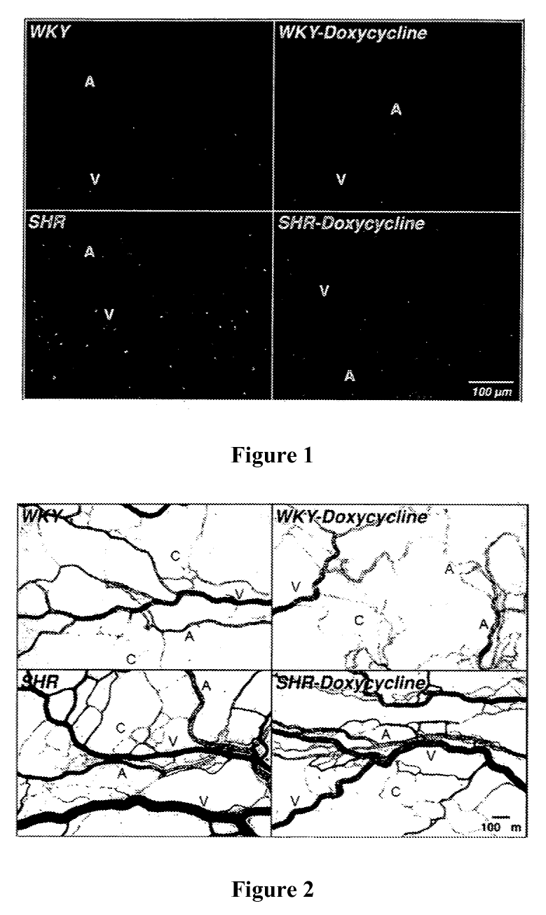

In order to show an example of the mechanism as described in this disclosure and the effects of a protease inhibitor on such mechanism, experiments have been performed on the spontaneously hypertensive rat (SHR). It has been shown that in the SHR, a model of essential hypertension in man, a previously undescribed mechanism exists that leads to organ injury and cardiovascular complications due to metalloproteinase activation (MMP 2, 9, and probably others). A group of SHR and its control strain, the Wistar Kyoto (WKY) rat, were treated for a period of 10-12 weeks with a broad acting MMP inhibitor (doxycyline, 55 mg/liter) in drinking water. The results are presented in FIGS. 1-3 and described in more detail below. FIG. 1 shows MMP2, 9 activity detected by fluorescent intensity (arrows) derived by cleavage of the fluorescent substrate indicator. Four cases are shown, normotensive Wistar Kyoto rats (WKY) and spontaneously hypertensive rats (SHR) before (left panels) and 12 weeks after MMP-inhibition with doxycycline (right panels). Note that the MMP activity in endothelial cells and tissue mast cells (arrows) is almost completely abolished after the MMP blockade. FIG. 2 shows bright field micrograph of the rat mesentery microcirculation with NBT formazan deposits before (left panel) and after (right panel) 12-week treatment with MMP inhibition. Note the dramatic reduction of the dark blue formazan deposits in all hierarchies of microvessels from arteriole (A) to capillaries (C) and venules (V). Crossbar=100 micrometer.

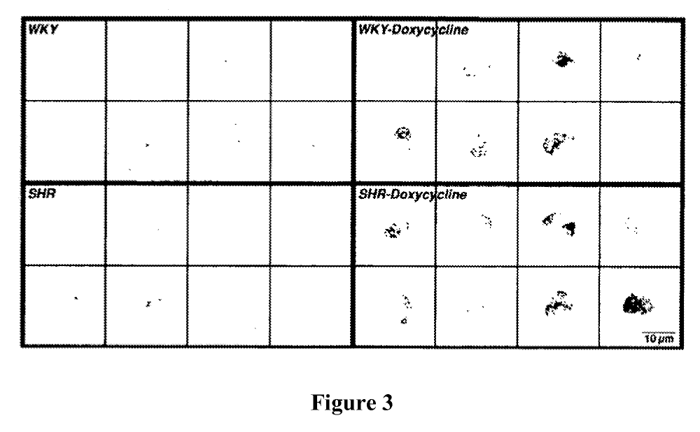

FIG. 3 shows a micrograph of fresh circulating leukocytes after labeling with CD18 integrin antibody (using Nova-Vector Red Avidin-Biotin substrate) against the extracellular domain before (left images) and after 12-week treatment with doxycycline (right) in mature WKY rats. Note that the usual cleavage of the integrin on the membrane of the cells is attenuated in the animals with MMP inhibition. The significant enhancement of the CD18 in the membrane of treated rats was confirmed by flow cytometry (data not shown). Crossbar=10 pm. In another experiment, and in order to explore the utility of chronic protease inhibition, a group of mature normotensive Wistar Kyoto rats (as well as a cohort of spontaneously hypertensive rats) were treated with a broad acting metalloproteases inhibitor (doxycyclin, 55 mg/liter in drinking water, average consumption—5.4 mg/kg/day) over a period of 12 weeks until the age of 30 weeks. Such treatment serves to significantly reduce the central blood pressure elevation observed over time in these animals (Table I), dramatically reduce the plasma protease activity (Table 2) and the MMP 2,9 tissue activity in the mesentery microcirculation, and reduce the superoxide production (by nitroblue tetrazolium detection, NBT) as well as the cleavage of the leukocyte adhesion integrin Mac1 (CD18). These results indicate that the enzymatic activity in plasma has the ability to cleave important membrane receptors including the insulin receptor, the aminoacid transporters for L-arginine to facilitate NO production and many others.

TABLE 1
Mean Arterial Blood Pressure (mmHg)
during Chronic MMP Inhibition

| | | Spontaneously |
|---|---|---|
| | | Hypertensive |
| Groups | Wistar Kyoto rat | rat |
| Control Rats | 134.4 ± 10.1 (3 rats) | 174.2 ± 1.7 (3 rats)* |
| After MMP inhibition | 95.7 ± 8.5 (5 rats) | 126.3 ± 13.5 (7 rats)* |
| (for 12 weeks) | | |

*p < 0.01 vs untreated control; Age = 30 weeks at time of measurement

TABLE 2
Plasma Protease Activity (FU)
During Chronic MMP Inhibition

| | | Spontaneously |
|---|---|---|
| | | Hypertensive |
| Groups | Wistar Kyoto rat | rat |
| Control Rats | 1764 ± 656 (3 rats) | 3289 ± 342 (3 rats)* |
| After MMP inhibition | 413 ± 40 (4 rats) | 472 ± 24 (7 rats)* |
| (for 12 weeks) | | |

*p < 0.03 vs untreated control; Age = 30 weeks at time of measurement

It is expected that similar treatment in other forms of diseases with metabolic syndrome x (diabetics, other forms of arterial, venous and pulmonary hypertension), will attenuate the inflammatory reaction in the circulation and thereby provide a significant clinical benefit against organ injury and organ failure. Such treatment could reduce the level of insulin resistance, enhance the fluid shear stress response, reduce blood pressure, reduce the tendency for blood clotting and thrombosis, enhance leukocyte adhesion to the endothelium and thereby restore the specific immune response and the acute repair to injury, and reduce apoptosis in hypertensives and diabetics and similar syndromes in aging.

Potential uses of the present invention are many. It may serve as alternative treatment for control of inflammation in essential hypertensive patients, patients with the metabolic syndrome x, in patients with aging hypertension, as well as type 1 and 2 diabetics. It includes improvement of insulin response, reduced thrombosis, reduced apoptosis and normalization of arterial dilation and immune response.

The following article is considered to be part of the present disclosure and is incorporated herein in its entirety: Kobayashi, N., DeLano, F. A., Schmid-Schonbein, G. W.: Oxidative stress promotes endothelial cell apoptosis and loss of microvessels in the spontaneous hypertensive rats.

The foregoing disclosure of the preferred embodiments of the present invention has been presented for purposes of illustration and description. It is not intended to be exhaustive or to limit the invention to the precise forms disclosed. This and all other referenced extrinsic materials are incorporated herein by reference in their entirety. Where a definition or use of a term in an incorporated reference is inconsistent or contrary to the definition of that term provided herein, the definition of that term provided herein applies and the definition of that term in the reference does not apply.

Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

In addition, the claims directed to the method and/or process of the present invention should not be limited to the performance of their steps in the order written, and one skilled in the art can readily appreciate that the sequences may be varied and still remain within the spirit and scope of the present invention.

Thus, specific embodiments and applications of uses of endopeptidases in treatment of disease have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of marketing, comprising:
providing a preparation of doxycycline for use by a patient population; and
placing the preparation in a stream of commerce with an expectation that at least some of the preparation will be used to treat a non-gingival disease associated with an matrix metalloproteinase (MMP) activity.

2. The method of claim 1, further comprising providing information to a medical provider regarding a correlation between doxycycline and the MMP activity.

3. The method of claim 1, further comprising providing information to a patient within the patient population regarding a correlation between doxycycline and the MMP activity.

4. The method of claim 1, wherein the expectation arises at least in part from receiving information from a scientist involved in establishing a correlation between doxycycline and the MMP activity.

5. The method of claim 1, wherein the expectation arises at least in part from an agent reading a journal article that discusses a correlation between doxycycline and the MMP activity.

6. The method of claim 1, further comprising subscribing to a peer-reviewed journal that contains an article that discusses a correlation between doxycycline and the MMP activity.

7. The method of claim 1, wherein the preparation comprises an orally administrable form selected from the group consisting of a pill, a capsule, a tablet, and a caplet.

8. The method of claim 1, wherein the preparation comprises a bottled drink.

9. The method of claim 1, further comprising marketing the preparation in a package that includes a reference to a correlation between doxycycline and the MMP activity.

10. The method of claim 9, wherein the reference is included in a package insert.

11. The method of claim 9, wherein the reference is mandated by a government agency.

12. The method of claim 1, wherein the disease has an inflammatory etiology.

13. The method of claim 1, wherein the disease has an etiology associated with insulin resistance.

14. The method of claim 1, wherein the disease has an etiology associated with free radical production.

15. The method of claim 1, wherein the disease has an etiology associated with hypertension.

16. The method of claim 1, wherein the step of placing is further performed with an understanding that treatment of the disease may include prophylactic treatment.
